(19) United States
(12) Patent Application Publication
(10) Pub. No.: US 2010/0092554 A1
(43) Pub. Date: Apr. 15, 2010

(54) COMBINATION WITH AN EXTENDED RELEASE TABLET FORMULATION CONTAINING PRAMIPEXOLE OR A PHARMACEUTICALLY ACCEPTABLE SALT THEREOF

(75) Inventors: Juergen Reess, Ulm (DE); Laurence Salin, Reims (FR)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM INTERNATIONAL GMBH, Ingelheim am Rhein (DE)

(21) Appl. No.: 12/596,894

(22) PCT Filed: Apr. 22, 2008
(86) PCT No.: PCT/EP08/54854

§ 371 Date: Nov. 10, 2009

(30) Foreign Application Priority Data
Apr. 24, 2007 (EP) 07106864.7

(51) Int. Cl. A61K 9/22; A61K 31/428; A61K 9/16

(52) U.S. Cl. 424/468; 514/367; 424/490

(57) ABSTRACT

The present invention is directed to a combination of an extended release tablet formulation containing pramipexole or a pharmaceutically acceptable salt thereof with a conventional treatment option of Parkinson's Disease.

### FIELD OF THE INVENTION

The present invention is directed to a combination of an extended release tablet formulation containing pramipexole or a pharmaceutically acceptable salt thereof with a conventional treatment option of Parkinson's Disease.

### BACKGROUND OF THE INVENTION

Pramipexole is a known dopamine D2 receptor agonist. It is structurally different from the ergot-derived drugs, e.g. bromocriptine or pergolide. It is also pharmacologically unique in that it is a full agonist and has receptor selectivity for the dopamine D2 family of dopamine receptors.

Pramipexole is designated chemically as (S)-2-Amino-4,5,6,7-tetrahydro-6-(propylamino)benzothiazole and has the molecular formula C₁₀H₁₇N₃S and a relative molecular mass of 211.33. The chemical formula is as follows:

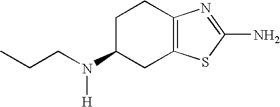

The salt form commonly used is pramipexole dihydrochloride monohydrate (molecular formula C₁₀H₂₁Cl₂N₃OS; relative molecular mass 302.27). Pramipexole dihydrochloride monohydrate is a white to off-white, tasteless, crystalline powder. Melting occurs in the range of 296° C. to 301° C., with decomposition. Pramipexole is a chiral compound with one chiral centre. Pure (S)-enantiomer is obtained from the synthetic process by chiral recrystallization of one of the intermediates during synthesis. The term pramipexole as used in this description shall comprise the base as well as any suitable pharmaceutically acceptable salt thereof, in particular the dihydrochloride or the dihydrochloride monohydrate thereof.

As commonly known, modified release of active ingredient(s) allows to simplify the patient's administration scheme by reducing the amount of recommended daily intakes, improves patient's compliance, and attenuates adverse events, e.g. related to high plasma peaks. Modified release pharmaceutical preparations regulate the release of the incorporated active ingredient or ingredients over time and comprise formulations with a controlled, a prolonged, a sustained, a delayed, a slow or an extended release, so they accomplish therapeutic or convenience objectives not offered by conventional dosage forms such as solutions or promptly dissolving dosage forms.

### DESCRIPTION OF THE INVENTION

The present invention relates to a combination of a conventional treatment of Parkinson's Disease and once daily extended (or slow) release formulation comprising pramipexole.

In the context of the present invention the most preferred pramipexole formulation corresponds to one according to WO2006/015942 or according to WO2006/015943, both of which herewith are incorporated by reference. The pramipexole extended release formulation allows for different release rate types dependent or independent from the pH value.

In the following the two extended release formulation will be outlined in principle.

The pramipexole containing formulation according to WO2006/015942 relates to an extended release tablet formulation comprising pramipexole or a pharmaceutically acceptable salt thereof in a matrix comprising at least one water swelling polymer other than pregelatinized starch.

Preferably the extended release tablet formulation of pramipexole comprises a matrix with at least two water swelling polymers other than pregelatinized starch, and wherein at least one of the at least two polymers is an anionic polymer.

Also preferred is an extended release tablet formulation of pramipexole, wherein the anionic polymer is selected from the group of optionally crosslinked acrylic acid polymers, methacrylic acid polymers, alginates, and carboxymethylcellulose.

Also preferred is an extended release tablet formulation of pramipexole, wherein the anionic polymer is an optionally crosslinked acrylic acid polymer, and wherein the content of the optionally crosslinked acrylic acid polymer in the matrix is from about 0.25 wt.-% to about 25 wt.-%, and preferably from about 0.5 wt.-% to about 15 wt.-%, and preferably from about 1 wt.-% to about 10 wt.-%.

Also preferred is an extended release tablet formulation of pramipexole, wherein at least one of the at least two polymers is a substantially neutral polymer other than pregelatinized starch.

Also preferred is an extended release tablet formulation of pramipexole, wherein the substantially neutral polymer is selected from hydroxypropylcellulose and hydroxypropylmethylcellulose.

Particularly preferred is an extended release tablet formulation of pramipexole, wherein the substantially neutral polymer is hydroxypropyl methylcellulose, and wherein the content of hydroxypropyl methylcellulose in the matrix is from about 10 wt.-% to about 75 wt.-% and preferably from about 25 wt.-% to about 65 wt.-%.

Particularly preferred is an extended release tablet formulation of pramipexole, wherein the matrix comprises about:

| | |
|---|---|
| (a) pramipexole or a salt thereof | 0.05 to 5 wt.-% |
| (b) anionic water swelling polymer(s) | 0.25 to 25 wt.-% |
| (c) neutral water swelling polymer(s) | 10 to 75 wt.-% |
| (d) further excipients | ad 100 wt.-% |

Particularly preferred is an extended release tablet formulation consisting of Pramipexole-dihydrochloride monohydrate, Hypromellose 2208, Corn starch, Carbomer 941, Colloidal silicon dioxide and Magnesium stearate.

A preferred embodiment of the pramipexole component of the present invention relates to an extended release tablet formulation comprising pramipexole or a pharmaceutically acceptable salt thereof in a matrix comprising
(a) at least one water swelling polymer other than pregelatinized starch and optionally excipients, the resulting tablet providing a pH-independent in vitro release characteristic in the range from pH 1 to 7.5, or
(b) at least one water swelling anionic polymer and optionally excipients, the resulting tablet providing a pH-dependent release characteristic with a faster release characteristic in the range of pH<4.5, and a slower and further on pH-independent release characteristic in the range from pH 4.5 to 7.5.

Most preferably the pramipexole component of the present invention relates to a matrix of the extended release tablet formulation comprising at least one water swelling polymer other than pregelatinized starch, preferably a water swelling essentially neutral polymer, a water swelling anionic polymer and optionally excipients, the resulting tablet providing a pH-dependent release characteristic with a faster release characteristic in the range of pH<4.5, and a slower and further on pH-independent release characteristic in the range from pH 4.5 to 7.

The extended release formulations of pramipexole according to the present invention intended for oral administration allow to select and estimate which in vitro release characteristic and timing of a formulation is most suitable to achieve the desired in vivo plasma profiles preferably with a once daily application. Therefore, a formulation principle with several variants has been developed for a single unit matrix tablet, i.e. formulations having different release rate types are provided and a different pH dependency is available. These alternative formulations are beneficial to patients as the extended release drug delivery will allow patients to treat their symptoms with a single daily dose, thereby increasing patient convenience and compliance.

The term “in vitro release characteristic” as used hereinbefore or hereinafter is directed to a release characteristic as obtained in a kind of normally used liquid medium for in vitro experiments wherein the release of active ingredient from the extended release formulation can occur, i.e. for example in in vitro dissolution media, but also in body fluids or simulated body fluids, more in particular in the gastro-intestinal fluids.

In the frame of the present invention the term “extended” release should be understood in contrast to an immediate release, the active ingredient is gradually, continuously liberated over time, sometimes slower or faster, dependent or independent from the pH value. In particular, the term indicates that the formulation does not release the full dose of the active ingredient immediately after oral dosing and that the formulation allows a reduction in dosage frequency, following the definition for extended release, interchangeable with slow release. A slow or extended release, used synonymously with prolonged action, sustained release, or modified release, dosage form is a dosage form that allows a reduction in dosing frequency or a significant increase in patient compliance or therapeutic performance as compared to that presented as a conventional dosage form (e.g. as a solution or an immediate drug-releasing, conventional solid dosage form).

A release characteristic which is pH-independent indicates that the release characteristic is virtually the same in different pH media.

According to the teaching of the present invention, extended release tablet formulations are provided with different in vitro release profiles.

The extended release tablets of the present invention are believed to apply a swelling and partly eroding polymer matrix. Based on the assumed mechanisms, the release profile may roughly follow a square root of time to exponential in vitro release characteristic. Depending on the particular embodiment formulation (a) is widely independent from the pH value in the range from pH 1 to 7.5, and formulation (b) is faster in simulated gastric juice having a pH<4.5 but are independent from the pH value in the range from 4.5 to 7.5. A faster release in simulated gastric juice versus slower release in the intestinal fluid can be advantageous in cases where a loading dose effect from the dosage form is desired, whereas a widely pH independent release profile can be advantageous to reduce the risk of dose dumping and food effects.

According to the present invention under “formulation (a)” is understood the tablet formulation wherein the matrix comprises the composition as above-defined under (a) and under “formulation (b)” is understood the tablet formulation wherein the matrix comprises the composition as above-defined under (b).

The water swelling polymer of the present invention represents at least one hydrophilic water swelling polymer constituting the extended release matrix which slowly releases the pramipexole or its salt as active ingredient. The polymer swells upon contact with aqueous fluid following administration, resulting in a viscous, drug release regulating gel layer. The viscosity of the polymer preferably ranges from 150 to 100,000 mPa·s (apparent viscosity of a 2% aqueous solution at 20° C.).

Examples of such polymers are water swelling substantially neutral polymers or water swelling anionic polymers.

The term “water swelling substantially neutral polymers” of the present invention comprises

alkylcelluloses, such as, methylcellulose; hydroxyalkylcelluloses, for example, hydroxymethylcellulose, hydroxyethylcellulose, hydroxypropylcellulose and hydroxybutylcellulose; hydroxyalkyl alkylcelluloses, such as, hydroxyethyl methylcellulose and hydroxypropyl methylcellulose; carboxyalkylcellulose esters; other natural, semi-synthetic, or synthetic di-, oligo- and polysaccharides such as galactomannans, tragacanth, agar, guar gum, and polyfructans; methacrylate copolymers; polyvinylalcohol; polyvinylpyrrolidone, copolymers of polyvinylpyrrolidone with vinyl acetate; combinations of polyvinylalcohol and polyvinylpyrrolidone; polyalkylene oxides such as polyethylene oxide and polypropylene oxide and copolymers of ethylene oxide and propylene oxide, preferably cellulose ether derivatives such as hydroxypropyl methylcellulose and hydroxypropyl cellulose, most preferred hydroxypropyl methylcellulose.

The term “water swelling anionic polymer” of the present invention comprises acrylic acid polymerisate, methacrylic acid copolymers, alginates, carrageenans, acacia, xanthan gum, chitin derivates such as chitosan, carmellose sodium, carmellose calcium, preferably acrylic acid polymerisate.

Different viscosity grades of hydroxypropyl cellulose and hydroxypropyl methylcellulose are commercially available. Hydroxypropyl methylcellulose (HPMC) preferably used in the present invention has a viscosity grade ranging from about 3,500 mPa·s to about 100,000 mPa·s, in particular ranging from about 4,000 mPa·s to about 20,000 mPa·s and most in particular a viscosity grade of about 6,500 mPa·s to about 15,000 mPa·s (apparent viscosity of a 2% aqueous solution at 20° C.), e.g. hypromellose 2208 or 2206 (DOW, Antwerp, Belgium). HPMC type 2208 contains 19-24% by weight methoxy and 4-12% by weight hydroxypropoxy substituents.

Hydroxypropyl cellulose having a viscosity higher than 1,500 mPa·s (apparent viscosity of a 1% aqueous solution at 20° C.) is preferred, in particular hydroxypropyl cellulose having a viscosity in the range from about 1500 to about 3000 mPa·s, preferably from 4000 to 6500 mPa·s (2% aqueous solutions), e.g. the Klucel series such as Klucel M (Hercules, Wilmington, USA).

Without wishing to be bound by theory, there are believed to exist three main mechanisms by which pramipexole or a salt thereof can be released from a hydrophilic matrix: dissolution, erosion and diffusion. Pramipexole or its salt will be released by the dissolution mechanism when it is homogeneously dispersed in a matrix network of a soluble polymer. The network will gradually dissolve in the gastrointestinal tract, thereby gradually releasing its load. The matrix polymer can also gradually be eroded from the matrix surface, likewise releasing pramipexole or its salt in time. When pramipexole is processed in a matrix made up of an insoluble polymer, it will be released by diffusion: the gastro-intestinal fluids penetrate the insoluble, sponge-like matrix and diffuse back out loaded with drug.

Therefore, the water swelling polymers constituting the matrix, particularly in a matrix according to formulation (a), mainly provide for the controlled pharmacokinetic release profile of the preparation. Depending on the amount of water swelling polymer(s) processed in the preparation, the release profile can be tuned, i.e. larger amounts of swelling polymer lead to a more pronounced sustained release effect and vice versa. Preferably, the amount of water swelling polymer in the present formulation ranges from about 10 to about 80% by weight.

In addition, when using a combination of polymers, the ratio of said polymers also influences the release profile of the preparation. A combination of different polymers offers the possibility of combining different mechanisms by which pramipexole is released from the matrix. Such combination facilitates control of the pharmacokinetic release profile of the preparation at will. For example, when using one or more water swelling polymers, in particular hydroxypropyl cellulose and hydroxypropyl methylcellulose, the weight percentage of hydroxypropyl methylcellulose preferably ranges from 25 to about 62%; the weight percentage of hydroxypropyl cellulose preferably ranges between 0% and about 16%.

Release of pramipexole or a salt thereof from a matrix containing hydroxypropyl cellulose and hydroxypropyl methylcellulose occurs by a combined set of release mechanisms. Due to the higher solubility of hydroxypropyl methylcellulose compared with hydroxypropyl cellulose, the former will gradually dissolve and erode from the matrix, whereas the latter will more act as a sponge-like matrix former releasing the active ingredient mainly by diffusion.

The extended release tablet formulation according to formulation (a) is pH-independent. Therefore, the disadvantage that food related dose-dumping may be encountered is avoided. The problem of food related dose-dumping in fed patients can be attributed to a lot of factors such as the mechanical forces that are exerted by the stomach on its content and thus on an ingested preparation as well as the different pH regions of the gastro-intestinal tract. Since the pH values encountered in the gastro-intestinal tract vary not only with the region of the tract, but also with the intake of food, an extended release formulation preferably also has to provide an extended release profile and in particular has to avoid dose-dumping regardless whether the patient is in fasted or fed conditions.

The pramipexole component of the present invention according to the oral extended release formulation (a) retains its pharmacokinetic release profile along its way through the gastro-intestinal tract so as to avoid undesirable fluctuations in drug plasma concentrations or complete dose-dumping, in particular avoids dose-dumping in different regions of the gastro-intestinal tract.

Beside pramipexole or a salt thereof, and the water swelling polymer(s), the formulation of the present invention may also optionally comprise further excipients, i.e. pharmaceutically acceptable formulating agents, in order to promote the manufacture, compressibility, appearance and taste of the preparation. These formulating agents comprise, for example, diluents or fillers, glidants, binding agents, granulating agents, anti-caking agents, lubricants, flavors, dyes and preservatives. Other conventional excipients known in the art can also be included.

The filler may be selected from soluble fillers, for example, sucrose, lactose, in particular lactose monohydrate, trehalose, maltose, mannitol and sorbitol. Different grades of lactose can be used. One type of lactose preferably used in the present invention is lactose monohydrate 200 mesh (DMV, Veghel, The Netherlands). Another lactose monohydrate, lactose monohydrate of the type DCL 11 (DMV, Veghel, The Netherlands), can also preferably be used. The notation DCL refers to “Direct Compression Lactose”. The number 11 is a reference number of the manufacturer. In case of a water soluble active ingredient, like the one described in this invention, more preferably water insoluble fillers, such as starch and starch derivates other than pregelatinized starch, e.g. corn starch, potato starch, rice starch or wheat starch, microcrystalline cellulose, dibasic calcium phosphate dihydrate and anhydrous dibasic calcium phosphate, preferably corn starch, can be used in addition or instead of the water soluble fillers. The total weight percentage of filler ranges between about 5% and about 75% by weight.

A glidant can be used to improve powder flow properties prior to and during tableting and to reduce caking Suitable glidants include colloidal silicon dioxide, magnesium trisilicate, powdered cellulose, talc, tribasic calcium phosphate and the like. Colloidal silicon dioxide is preferably included as a glidant in an amount up to about 2%, preferably about 0.2% to about 0.8%, by weight of the tablet.

A lubricant can be used to enhance release of a tablet from apparatus on which it is formed, for example by preventing adherence to the face of an upper punch (“picking”) or lower punch (“sticking”). Suitable lubricants include magnesium stearate, calcium stearate, canola oil, glyceryl palmitostearate, hydrogenated vegetable oil, magnesium oxide, mineral oil, poloxamer, polyethylene glycol, polyvinyl alcohol, sodium benzoate, sodium lauryl sulfate, sodium stearyl fumarate, stearic acid, talc, hydrogenated vegetable oil, zinc stearate and the like. In one embodiment, magnesium stearate is included as a lubricant in an amount of about 0.1% to about 1.5%, preferably about 0.3% to about 1%, by weight of the tablet.

Among the optional formulating agents that further may be comprised in the matrix formulation there may be mentioned agents such as polyvidone; copovidone; starch; acacia; gelatin; seaweed derivatives, e.g. alginic acid, sodium and calcium alginate; cellulose, preferably microcrystalline cellulose, cellulose derivatives, e.g. ethylcellulose, hydroxypropylcellulose, hydroxypropylmethylcellulose, having useful dry or wet binding and granulating properties; and antiadherents such as talc and magnesium stearate.

According to a preferred embodiment of the present invention the matrix of the extended release tablet formulation of alternative (a) comprises or essentially consists of hydroxypropyl methylcellulose, such as hypromellose, and further excipients. The amount of hydroxypropyl methylcellulose is preferably in the range from 10 to 75%, particularly preferred from 25 to 65% most preferred from 35 to 55% by weight. The amount of further excipients is preferably in the range from 90 to 25%, particularly preferred from 75 to 35%, most preferred from 65 to 45% by weight.

The expression “consisting essentially” is understood in the sense that it does not in principle exclude the presence, in addition to the mandatory components mentioned, of other components, the presence of which does not affect the essential nature of the formulation.

In some embodiments of the present invention it is provided a pH-dependent release profile, the release of pramipexole or its salt from the tablet and subsequent the absorption into the blood stream can vary during the passage of the dosage form along the gastro-intestinal tract. Thus, formulation (b) provides a pH-dependent release characteristic wherein the release characteristic in the range of pH<4.5 is faster and a slower and further on pH-independent release characteristic in the range from 4.5≦pH≦7.5.

The above details for the water swelling polymer and selection and type of optional excipients may apply to formulation (b), too.

Moreover, an anionic water swelling polymer, preferably an acrylic acid polymerisate is mandatorily present in formulation (b), which is preferably selected from carbomer or Carbopol® series, known acrylic acid polymerisates having high molecular weights. Particularly preferred are for example carbomer 941 (Carbopol® 71 G, Carbopol® 971) and carbomer 934 (Carbopol® 974). The acrylic acid polymerisate is preferably present in the range of 0.25 to 25% by weight, particularly preferred 0.5 to 15% by weight, most preferred 1 to 10% by weight. The pH dependency of formulation (b) results from the presence of an anionic water swelling polymer, particularly preferred from the presence of acrylic acid polymerisate which intends to swell in a greater extent in the acid pH range above pH 4.5 and in the alkaline pH range.

An increasing amount of acrylic acid leads to a decrease of the release rate. Therefore, adjusting the amount of acrylic acid polymerisate makes it possible to further tune the dissolution profiles as desired. To adjust the amount of acrylic acid polymerisate in the preferred range from 0.25 to 25% by weight provides the further advantage that the desired, resp. matching, dissolution profiles can be adjusted, resp. maintained, for a variety of formulations composed of different amounts and/or types of gel-forming agents, water swelling polymers, fillers, and dry binders.

According to a preferred embodiment of the present invention the matrix of the extended release pramipexole tablet formulation comprises or essentially consists of hydroxypropyl methylcellulose, acrylic acid polymerisate and further excipients. The amount of hydroxypropyl methylcellulose is preferably in the range from 10 to 75%, particularly preferred from 25 to 65%, most preferred from 35 to 55% by weight. The amount of acrylic acid polymerisate is preferably as above-mentioned. The amount of additional excipients is preferably in the range from 90 to 25% particularly preferred from 75 to 35%, most preferred from 65 to 45% by weight. Optionally carboxymethylcellulose sodium may additionally be present preferably in the range from 5 to 50%, particularly preferred from 10 to 40%, most preferred from 15 to 30% by weight.

As active ingredient, pramipexole or a pharmaceutically acceptable salt thereof, may be present in any amount suitable for the desired treatment of a patient. A preferred salt of pramipexole is the dihydrochloride salt, most preferably in the form of the monohydrate. Usual amounts are from about 0.1 to about 5 mg pramipexole salt. According to a particularly preferred embodiment e.g. 0.750 mg pramipexole dihydrochloride monohydrate, corresponding to 0.524 mg anhydrous base, is used in the extended release tablet formulation according to the present invention. However, any other amount of active ingredient suitable for treatment may be used with the only proviso that the amount of pramipexole or salt is sufficient to provide a daily dose in one to a small plurality, for example one to about 4, of tablets to be administered at one time. Preferably the full daily dose is delivered in a single tablet. An amount of pramipexole salt, expressed as pramipexole dihydrochloride monohydrate equivalent, of about 0.1 to about 10 mg per tablet, or about 0.05% to about 5% by weight of the composition, will generally be suitable. Preferably an amount of about 0.2 to about 6 mg, more preferably an amount of about 0.3 to about 5 mg, per tablet is present. Specific dosage amounts per tablet e.g. include 0.375, 0.5, 0.75, 1.0, 1.5, 3.0 and 4.5 mg pramipexole dihydrochloride monohydrate. The amount that constitutes a therapeutically effective amount varies according to the condition being treated, the severity of said condition, and the patient being treated.

An extended release tablet formulation suitable as the pramipexole component in the combination according to the present invention, has preferably the following composition:

| | |
|---|---|
| pramipexole or a salt thereof | 0.05 to 5% by weight |
| water swelling polymer(s) | 10 to 75% by weight |
| acrylic acid polymerisate | 0 to 25% by weight |
| optional further excipient(s) | ad 100% by weight. |

Therefore, a particularly preferred extended release tablet formulation of the pramipexole component of present invention consists of

0.1 to 2% by weight of pramipexole or a salt thereof;
25 to 65% by weight of hydroxypropyl methylcellulose;
0 to 40% by weight of carboxymethylcellulose sodium;
0 to 75% by weight of corn starch other than pregelatinized starch;
0 to 15% by weight of acrylic polymerisate, preferably carbomer 941;
0.5 to 50% by weight of excipients, preferably selected from the group consisting of colloidal silicon dioxide, magnesium stearate, lactose monohydrate, mannitol, microcrystalline cellulose, dibasic anhydrous calcium phosphate, hydroxyproylcellulose, povidone, copovidone, talc, macrogols, sodium dodecylsulfate, iron oxides and titanium dioxide.

Starch other than pregelatinized starch, preferably corn starch if present, may impart several functions at the same time such as filler, glidant, and the like. However, it may be preferred to exclude starch completely from the tablet formulation according to the present invention, which may be replaced by one or more of the above-mentioned other excipient(s). Furthermore, a starch having a tensile strength of at least about 0.15 kN cm⁻² at a solid fraction representative of the tablet as claimed according to WO 2004/010997 is not required according to the present invention.

It is preferred that no coating is present on the tablet formulation according to the present invention. However, the extended release tablet of the invention may comprise a nonfunctional coating. A nonfunctional coating can comprise a polymer component, for example HPMC, optionally with other ingredients, for example one or more plasticizers, colorants, etc. The term “nonfunctional” in the present context means having no substantial effect on release properties of the tablet, and the coating serves another useful purpose. For example, such a coating can impart a distinctive appearance to the tablet, provide protection against attrition during packaging and transportation, improve ease of swallowing, and/or have other benefits. A nonfunctional coating should be applied in an amount sufficient to provide complete coverage of the tablet. Typically an amount of about 1% to about 10%, more typically an amount of about 2% to about 5%, by weight of the tablet as a whole, is suitable.

The pramipexole tablets of the present invention can be of any suitable size and shape, for example round, oval, polygonal or pillow-shaped, and optionally bear nonfunctional surface markings. According to the present invention it is preferred that the extended release tablets are white to off-white and of oval or round, biconvex, shape.

The pramipexole containing formulation according to WO2006/015943 also is a once daily formulation comprising extended (or slow) release pellets and again two alternative formulation principles allow different release rate types dependent or independent from the pH value.

One embodiment of this aspect of the present invention relates to an extended release pellet comprising an active ingredient selected from pramipexole and the pharmaceutically acceptable salts thereof, and at least one release-modifying excipient.

Preferably in the pellets the active ingredient pramipexole is embedded within a matrix formed by the at least one release-modifying excipient, which is preferably selected from the group of lipids, waxes, and water-insoluble polymers.

Also preferred is an extended release pellet comprising a core and a coating, wherein at least one release-modifying excipient is incorporated in the coating.

Also preferred is an extended release pellet, wherein the active ingredient is incorporated in the core.

Also preferred is an extended release pellet, wherein the coating comprises at least a first layer and a second layer surrounding the first layer, wherein the first layer comprises the active ingredient, and wherein the second layer comprises at least one release-modifying excipient, preferably selected from ethylcellulose, cellulose acetate, polyvinylacetate, polyacrylates, polymethacrylates, and ammonio methacrylate copolymer.

Most preferred is an extended release pellet, wherein the second layer further comprises at least one water-soluble excipient, preferably selected from hydroxypropylcellulose, hydroxypropyl methylcellulose, polyvinylpyrrolidone and polyethylene glycol.

Particularly preferred is an extended release pellet, wherein the second layer further comprises an enteric-coating polymer, preferably selected from methacrylic acid copolymers type A and B.

Particularly preferred is an extended release pellet, wherein the second layer comprises from about 10 to about 85 wt.-% of the enteric-coating polymer and from about 15 to about 75 wt.-% of the water-insoluble polymer.

More particularly preferred is an extended release pellet, wherein the core comprises a saccharide, such as saccharose, starch, cellulose, and a cellulose derivative, preferably microcrystalline cellulose.

In a further embodiment the present invention relates to an extended release pellet formulation comprising
an inert pellet core
a first layer being an active ingredient layer comprising pramipexole or a pharmaceutically acceptable salt thereof and optionally one or more wet binders and other excipients; and
a second layer provided on the first layer, the second layer being an extended release coating comprising
   (1) at least one water-insoluble polymer and optionally a pore former, the resulting pellet having a pH-independent in vitro release characteristic or
   (2) a mixture of a pH-dependent enteric-coating polymer and a pH-independently water swelling polymer, the resulting pellet having a close to zero order in vitro release characteristic at acidic pH values up to pH 6.8, an accelerated release above pH 6.8 and a more accelerated release above pH 7.3.

The expression “layer” should be understood in its broadest sense also including a coating or a film or any kind of (partly or fully) surrounding material used in the pharmaceutical sector and having a defined thickness.

Instead of using an inert pellet core and a 1^{st} layer of active principle, pellets can also be formed by extrusion of active principle together with excipients in a wet extrusion or melt extrusion process.

The extended release formulations (1) and (2) according to the present invention intended for oral administration allow to select and estimate which in vitro release characteristic and timing of a formulation is most suitable to achieve the desired in vivo plasma profiles preferably with a once daily application. Therefore, two different formulation principles have been developed for pellets. The two formulation principles have different release rate types and a different pH dependency is available. These alternative formulations are beneficial to patients as the extended release drug delivery will allow patients to treat their symptoms with a single daily dose, thereby increasing patient convenience and compliance.

The terms “in vitro release characteristic” or “extended release” or “consisting essentially” have been defined above.

According to the teaching of the present invention two types of extended release pellet formulations are available showing different in vitro release characteristics. The two types have the same structure, i.e. an inert pellet core and a first and a second layer applied thereon in this order, the first layer represents the active ingredient layer comprising pramipexole or a pharmaceutically acceptable salt thereof and optionally a binder and further excipients, the second layer represents a functional coating either comprising a water-insoluble polymer with a pore former or a mixture of an enteric-coating polymer, i.e. which is resistent against gastric juice, and a non-dissolving water swelling polymer.

According to the present invention under “formulation (1)” is understood the pellet formulation having the second layer as above-defined under (1) and under “formulation (2)” is understood the pellet formulation having the second layer as above-defined under (2) whereas the inert pellet core and first layer compositions of formulation (1) and (2) will be the same.

The extended release pellet formulation (1) of the present invention applies a water-insoluble polymer preferably with a pore former in the second layer leading to an exponential (1^{st} order) in vitro release characteristic, which is widely independent of the pH value. The extended release pellet formulation (2) of the present invention applies a mixture of a pH-dependent enteric-coating polymer and a pH-independently water swelling polymer, the resulting layer having a close to zero order in vitro release characteristic over a broad period of time at acidic pH values up to pH 6.8, an accelerated release above pH 6.8 and an more accelerated release above pH 7.3. In addition to the close to zero order release for the main portion of drug, the latter is furthermore characterized by a certain lag time until drug release becomes substantial and, after the main portion of drug is released, by a flattening of the release profile until an asymptote is reached. This results in a sigmoide profile, i.e. a s-shaped dissolution profile.

A close to zero order in vitro release characteristic indicates a curve which has a virtually constant ascending slope.

The inert pellet core present in both alternate pellet formulations (1) and (2) of the present invention comprises saccharides, preferably polysaccharides, cellulose or a cellulose derivative, starch and/or waxes. It is preferred if the core consists of or essentially consists of a saccharide, preferably polysaccharide, or cellulose, particularly preferred saccharose or microcrystalline cellulose. Most preferred is microcrystalline cellulose. The size of the cores may be sieve fractions between 0.1 and 3.0 mm, preferably between 0.5 and 1.5 mm.

In case the inert pellet core consists or essentially consists of microcrystalline cellulose it has been found that the thickness of the second layer applied thereon may be decreased to a great extent compared to the use of other core materials, e.g. if the core is composed of saccharose. Therefore, the amount of release controlling polymeric agents and overall spray volumes as well as process times to apply the coating dispersions or solutions may be reduced significantly while the release profile for the active ingredient may be maintained. The related advantages are reducing the amount of excipient and solvent materials used, reducing the process times and the embodiment is cost-saving.

The expression “consisting essentially” is understood in the sense that it does not in principle exclude the presence, in addition to the mandatory components mentioned, of other components, the latter can be excipients, the presence of which does not affect the essential nature of the formulation.

According to pellet formulations (1) and (2) of the present invention there is provided a first layer or coating on the inert core pellet comprising pramipexole or a pharmaceutically acceptable salt thereof and optionally one or more binders and further excipients. The first layer or coating normally has a thickness of 0.5 to 25 μm, preferably 1 to 5 μm.

As active ingredient pramipexole or a pharmaceutically acceptable salt thereof may be present in any amount suitable for the desired treatment of a patient. A preferred salt of pramipexole is the dihydrochloride salt, most preferably in the form of the monohydrate. Usual amounts within one dosage unit are from about 0.1 to about 5 mg pramipexole salt. According to a preferred embodiment e.g. 0.750 mg pramipexole dihydrochloride monohydrate, corresponding to 0.524 mg anhydrous base, is used in the extended release capsule or tablet formulation according to the present invention taking into account that all pellets which are filled in a capsule or compressed into a tablet are to give the desired dose strengths. Preferably the extended release pellets are filled into hard capsules, but also compressing of the pellets together with further excipients into tablets is possible.

However, any other amount of active ingredient suitable for treatment may be used with the only proviso that the amount of pramipexole or salt, that is the whole number of pellets being present in one capsule, is sufficient to provide a daily dose in one to a small plurality, for example one to about 4, of capsules to be administered at one time. Preferably the full daily dose is delivered in a single capsule. An amount of pramipexole salt, expressed as pramipexole dihydrochloride monohydrate equivalent, of about 0.1 to about 10 mg per capsule, or about 0.05% to about 5% by weight of the composition, will generally be suitable. Preferably an amount of about 0.2 to about 6 mg, more preferably an amount of about 0.3 to about 5 mg, per capsule is present. Specific dosage amounts per capsule e.g. include 0.375, 0.5, 0.75, 1.0, 1.5, 3.0 and 4.5 mg pramipexole dihydrochloride monohydrate. The amount that constitutes a therapeutically effective amount varies according to the condition being treated, the severity of said condition, and the patient being treated.

The binder(s) present in the first layer may be any suitable wet binder(s) as used in the pharmaceutical sector. Examples are hydrophilic polymers which may swell and glue upon contact with water. The viscosity of the polymers preferably ranges from 1 to 1,000 mPa·s (apparent viscosity of a 2% aqueous solution at 20° C.). Examples of such polymers are alkylcelluloses, such as, methylcellulose; hydroxyalkylcelluloses, for example, hydroxymethylcellulose, hydroxyethylcellulose, hydroxypropylcellulose and hydroxybutylcellulose; hydroxyalkyl alkylcelluloses, such as, hydroxyethyl methylcellulose and hydroxypropyl methylcellulose; carboxyalkylcelluloses, such as, carboxymethylcellulose; alkali metal salts of carboxyalkylcelluloses, such as, sodium carboxymethylcellulose; carboxyalkylalkylcelluloses, such as, carboxymethylethylcellulose; carboxyalkylcellulose esters; other natural, semisynthetic, or synthetic polysaccharides, such as, alginic acid, alkali metal and ammonium salts thereof, carrageenans, galactomannans, tragacanth, agar-agar, gummi arabicum, guar gummi, xanthan gummi, starches, pectins, such as sodium carboxymethylamylopectin, chitin derivates such as chitosan, polyfructans, inulin; polyacrylic acids and the salts thereof; polymethacrylic acids and the salts thereof, methacrylate copolymers; polyvinylalcohol; polyvinylpyrrolidone, copolymers of polyvinylpyrrolidone with vinyl acetate; combinations of polyvinylalcohol and polyvinylpyrrolidone; polyalkylene oxides such as polyethylene oxide and polypropylene oxide and copolymers of ethylene oxide and propylene oxide.

Preferable binders are polysaccharides, in particular cellulose derivatives and more preferred cellulose ether derivatives. A most preferred cellulose ether derivative is hydroxypropyl cellulose.

Different viscosity grades of hydroxypropyl cellulose and hydroxypropyl methylcellulose are commercially available. Hydroxypropyl methylcellulose preferably used as a wet binder in the present invention has a viscosity grade ranging from about 3 mPa·s to about 1,000 mPa·s, in particular ranging from about 3 mPa·s to about 20 mPa·s and preferably a viscosity grade of about 4 mPa·s to about 18 mPa·s (apparent viscosity of a 2% aqueous solution at 20° C.), e.g. hypromellose 2910 (DOW, Antwerp, Belgium).

Hydroxypropyl cellulose having a viscosity lower than 1,500 mPa·s (apparent viscosity of 1% aqueous solution at 20° C.) is preferred, in particular hydroxypropyl cellulose having a viscosity in the range from about 75 to about 150 mPa·s (5% aqueous solution), preferably from 300 to 600 mPa·s (10% aqueous solution), e.g. Klucel EFO (Hercules, Wilmington, USA).

Preferably, the amount of binder in the first layer of the pellet formulations (1) and (2) of the present invention ranges from 0 to about 30% by weight, preferably from about 10 to about 20% by weight. Also, a combination of binders may be used.

According to a preferred embodiment of this type of the pramipexole component the first layer of the extended release pellet formulation of alternatives (1) and (2) comprises or consists of hydroxypropyl cellulose, pramipexole or a pharmaceutically acceptable salt thereof and excipients. The amount of hydroxypropyl cellulose is preferably in the range from 1 to 30, particularly preferred from 5 to 25, most preferred from 10 to 20% by weight. The amount of excipients is preferably in the range from 1 to 40, particularly preferred from 2 to 25, most preferred from 5 to 15% by weight.

Beside pramipexole or a salt thereof, and the binder(s), the first layer or coating of both formulations (1) and (2) of the present invention may also optionally comprise excipients, i.e. pharmaceutically acceptable formulating agents, in order to promote the manufacture and coating properties of the preparation. These formulating agents comprise, for example, glidants, antiadherents, binding agents, granulating agents, anti-caking agents, and lubricants. Other conventional excipients known in the art can also be included.

A glidant and antiadherent can be used to improve the manufacturing during the spray process and to prevent sticking and picking of the pellets to each other. Suitable glidants include colloidal silicon dioxide, magnesium trisilicate, powdered cellulose, starch, talc, tribasic calcium phosphate and the like. In a preferred embodiment, talc is included as a glidant/antiadherent in an amount up to about 25%, preferably about 5% to about 15%, by weight of the first layer.

Waxes, lipids and water insoluble polymers may be used as release modifying agents. Suitable waxes include compounds that are chemically defined as esters of fatty acids and fatty alcohols or sterols, as well as derivatives and functional analogues thereof. Usually, the chain length of the fatty acid moiety is at least about 8 carbon atoms, and more typically at least about 12 carbon atoms. Waxes are plastic solids at room temperature, but very often have a moderately low melting point, such as below about 80-100° C. Waxes are usually somewhat more brittle than solid fats, and less greasy. More recently, also compounds which are chemically different from this definition but similar in their properties have been referred to as waxes. These waxes or functional analogues may also be used according to the present invention. Examples of potentially suitable waxes and wax analogues include white and yellow beeswax, carnauba wax, microcrystalline wax, spermaceti wax, candellila wax, saturated fatty acid esters, sugar cane wax, paraffin wax, castor wax, and wax mixtures such as nonionic or anionic emulsifying wax, cetyl esters wax, and lanolin. Among the presently preferred waxes are beeswax, carnauba wax, saturated fatty acid esters, and microcrystalline wax. Suitable lipids include lipophilic compounds or mixtures of natural or synthetic origin that have similar properties as glycerides and other natural lipids, such as phospholipids, sphingolipids, ceramides, sterols, steroids, and carotenoids. Lipids may be solid or liquid at room temperature, and may be viscous in their liquid state. Preferably, a lipid used to carry out the invention is solid at room temperature, even though a liquid lipid may also be used in mixtures, such as in a mixture with a solid lipid or wax. Examples of lipids which may be found useful include mono-, di- and glycerides of saturated or unsaturated fatty acids, such as—optionally hydrated or partially hydrated—vegetable oils (e.g. peanut, castor, coconut, cottonseed, palm, soybean), edible fat, hard fat, glyceryl behenate, glyceryl stearate, glyceryl palmitate; fatty acids such as stearic acid, behenic acid, palmitic acid, oleic acid, lauric acid, myristic acid, arachidic acid, linolenic acid, linoleic acid, arachidonic acid, and erucic acid; fatty alcohols such as those corresponding to the previously mentioned fatty acids, in particular cetyl alcohol, stearyl alcohol, oleyl alcohol, and palmityl alcohol; glycerides, fatty acids, or fatty alcohols which are modified with sorbitan or polyoxyethylene; and phospholipids such as lecithin or phosphatidylcholin. Particularly suitable lipids are solid or at least partially hydrated triglycerides including edible fat, hard fat, hydrated peanut-, castor-, coconut-, cottonseed-, palm-, and soybean oil, glyceryl behenate, glyceryl stearate, glyceryl palmitate, stearic acid, behenic acid, and palmitic acid.

Suitable water-insoluble polymers may comprise the water-insoluble polymers as defined below for the formulations according to the present invention.

Among the optional formulating agents that further may be comprised in the pellet formulation there may be mentioned agents such as polyvidone; starch; acacia gum; gelatin; seaweed derivatives, e.g. alginic acid, sodium and calcium alginate; cellulose, preferably microcrystalline cellulose, cellulose derivatives, e.g. ethylcellulose, hydroxypropylmethylcellulose, having useful binding and granulating properties.

According to the pellet formulation (1) of the present invention the second layer is provided on the first layer, the second layer, a functional layer, being an extended release coating or film coating comprising at least one water-insoluble polymer and preferably a pore former, the resulting pellet having an pH-independent in vitro release characteristic. Therefore, the second layer is a non soluble diffusion lacquer with pores leading to an exponential (1^{st} order) release profile of the pellet formulation (1) which has practically a pH-independent in vitro release characteristic. A release characteristic which is pH-independent indicates that the release characteristic is virtually the same in different pH media.

The water-insoluble polymer is defined as a polymer having a water solubility which is lower than 1 part soluble in 1,000, preferably lower than about 1 part soluble in 10,000 parts of solvent.

The release-controlling second layer, coating or film according to pellet formulation (1) comprises one or more hydrophobic or water-insoluble polymers such as cellulosic polymers e.g., methylcellulose, ethylcellulose, hydroxyethylcellulose, cellulose esters such as cellulose acetate, polyvinyl acetate, polymers and copolymers of acrylic acid and methacrylic acid and esters thereof, such as ammonio methacrylate copolymer, type B, and the like. Particularly preferred is ethylcellulose.

The hydrophobic or water-insoluble component, preferably ethylcellulose, typically constitutes about 1% to about 25%, preferably about 3% to about 10%, by weight of the pellet as a whole, provided that microcrystalline cellulose pellets are used as described above. In case sugar pellets are used higher amounts of ethylcellulose can become necessary.

The second layer can contain one or more pore formers, such as more water soluble polymers, like hydroxypropylcellulose, hydroxypropylmethylcellulose, and highly water soluble polymers, like polyvinyl pyrrolidone and polyethylene glycol, or other water soluble excipients, such as lactose and mannitol. Particularly preferred pore formers are polyethylene glycols (e.g. Macrogol 6000). The amount of pore former is suitably up to 40 percent by weight of the layer, coating or film, preferably up to 25% by weight. Pore formers like polyethylene glycols also serve as plasticizers, i.e. the function of such excipients either as plasticizer and/or pore former can not be clearly differentiated.

The second layer can optionally contain additional pharmaceutically acceptable excipients as mentioned above, preferably used are plasticizers, dyes and antiadherents. Particularly preferred plasticizers are polyethylene glycols (e.g. Macrogol 6000), triacetin, and triethylcitrate. The amount of plasticizer is suitably up to 25 percent by weight of the layer, coating or film. Anti-adherents, such as talc, and magnesium stearate can be used.

The extended release pellet formulation according to formulation (1) is pH-independent. Therefore, the disadvantage that food related dose-dumping which may be encountered is avoided. The problem of food related dose-dumping in fed patients can be attributed to a lot of factors such as the mechanical forces that are exerted by the stomach on its content and thus on an ingested preparation as well as the different pH regions of the gastro-intestinal tract. Since the pH values encountered in the gastro-intestinal tract vary not only with the region of the tract, but also with the intake of food, an extended release formulation preferably also has to provide a controlled release profile and in particular has to avoid dose-dumping regardless whether the patient is in fasted or fed conditions.

Therefore, the oral extended release formulation (1) according to the present invention retains its pharmacokinetic release profile along its way through the gastro-intestinal tract so as to avoid undesirable fluctuations in drug plasma concentrations or complete dose-dumping, in particular avoids dose-dumping in different regions of the gastro-intestinal tract.

The alternate pellet formulation (2) has the same structure with regard to the inert pellet core and first layer composition as defined for formulation (1) but a different second layer or functional film coating composition. Thus, the second layer of formulation (2) comprises or essentially consists of a mixture of a pH-dependent enteric-coating polymer and a pH-independently water swelling polymer, the resulting pellet having a close to zero order in vitro release characteristic at acidic pH values up to pH 6.8, an accelerated release above pH 6.8 and a more accelerated release above pH 7.3.

The pH-dependent enteric-coating polymer is preferably an anionic polymer, more preferably an anionic carboxylic acrylic polymer soluble above a pH value of 5.5, preferably above a pH value of 7.0. By an anionic polymer is meant a polymer containing anionic groups after dissociation depending on pH. For the purpose of this invention such polymer should be soluble above pH 5.5, preferably above pH 7.0. Preferably the anionic carboxylic acrylic polymer is selected from partly methyl esterified methacrylic acid polymers. Suitable partly methyl esterified methacrylic acid polymers are sold under the names Eudragit L and Eudragit S, preferably used are Eudragit 5100 and L100.

The water-insoluble, pH-independent swelling polymer is preferably selected from quarternary ammonium substituted acrylic polymers. Such polymers are sold under the names Eudragit RS and Eudragit RL having an ammonium substitution of about 5 and about 10 percent by weight, respectively. Preferably Eudragit RS 100 is used.

It is especially preferred if the layer or film coating comprises the enteric-coating polymer such as the anionic carboxylic acrylic polymer in an amount of 10 to 85 percent by weight of the layer or coating and the water-insoluble, pH-independent swelling polymer, selected from quarternary ammonium substituted acrylic polymers, in an amount of 15 to 75 percent by weight of the layer or coating. Depending on the amount and ratio of polymers processed in the preparation, the release profile can be tuned with regard to the release rate, that is the time to e.g. reach a level of 50% of drug dissolved, and with regard to the extent of pH dependency. In general, an excess of the anionic carboxylic acrylic polymer, e.g. Eudragit S 100, over the quarternary ammonium substituted acrylic polymers is required to achieve the desired accelerated dissolution characteristic at a pH above 6.8.

The second layer, coating or film normally has a thickness of 5 to 80 μm, preferably 20 to 60 μm.

The second functional layer according to formulation (2) of the present invention takes advantage of the fact that the time of passage through the small intestine is rather constant, said time is about 2 to 5 hours. According to the invention the change of pH from acid to about neutral at the pylorus is employed as a trigger mechanism changing the physical condition of the layer and finally causing the accelerated release of the active substance. Therefore the formulation releases a major part of its drug contents in the small intestine, and in the lower part of the intestinal system preferentially in the large intestine, i.e. the colon. With a layer or coating according to formulation (2) the release of pramipexole or a pharmaceutically acceptable salt thereof can be accelerated in the lower parts of the intestine, that is under conditions of higher physiological pH, thereby reducing the loss in bioavailability and increase in variability typically observed with pH independent release systems in situations of shorter gastrointestinal transit times.

According to a preferred embodiment of the present invention a pore-forming component may be present in the second layer or film coating of formulation (2). The pore-forming component may be selected from the group consisting of water soluble polymers, such as polyethylen glycols, polyvinyl pyrrolidon, and cellulose derivatives, such as hydroxypropyl cellulose and hydroxypropyl methylcellulose, preferably hydroxypropyl cellulose, preferably from the Klucel series. The pore-forming component is typically present in an amount of about 1% to about 25%, preferably about 2% to about 10%, by weight of the polymer mixture in the second layer.

A particular preferred pore-forming component is hydroxypropyl cellulose having a viscosity in the range from about 150 to about 700 mPa·s, preferably from 200 to 600 mPa·s, e.g. selected from the Klucel series such as Klucel EF or LF (Hercules, Wilmington, USA).

The polymer pore-forming component forms diffusion pores and leads to an accelerated hydration and an altering of the rebuffering characteristics of the layer or film coating with a change from acid to alkaline medium and results in an accelerated penetrability of the layer or coating for the active ingredient pramipexole or its salt in the pH range >7.3

Therefore, the presence of a pore-forming component provides the further advantage that the release characteristic is accelerated and occurs more rapid, i.e. the effects of the second layer are enhanced significantly.

According to a preferred embodiment an extended release pellet formulation has the following composition:

### Inert Pellet Core:

| | |
|---|---|
| saccharose or microcrystalline cellulose | 90 to 100% by weight |
| excipient(s) | 0 to 10% by weight |

### First Layer:

| | |
|---|---|
| pramipexole or a salt thereof | 50 to 100% by weight |
| binder(s) | 0 to 30% by weight |
| excipient(s) | 0 to 50% by weight |

### Second Layer:

| | |
|---|---|
| water-insoluble polymer(s) | 50 to 99% by weight |
| excipient(s) | 1 to 50% by weight |
| or | |
| a mixture of | |
| a pH-dependent enteric-coating polymer | 10 to 85% by weight |
| and | |
| a pH-independently water swelling polymer | 15 to 75% by weight |
| excipient(s) | 1 to 50% by weight |

The first and second layers or coatings should be applied at as uniform a thickness as possible to provide optimum control of release rate of the pramipexole or pramipexole salt.

If pellets are formed by extrusion, the following compositions are most suitable:

### Wet Extrusion:

Microcrystalline cellulose, powdered cellulose or starch is mixed with Pramipexole in ratios delivering the necessary amount of drug in a suitable number of pellets with regard to reproducibility of filling and acceptable capsule size. Extrusion is achieved by addition of water only or of water containing binders such as povidone or methylcellulose, hydroxypropylcellulose. In order to achieve the desired release rates, other excipients such as lactose, microcrystalline cellulose, starch etc. can be added.

### Melt Extrusion:

Melt extrusion is achieved either by hydrophilic or lipophilic compounds with melting points between 40 and 120° C. Suitable examples are polyethylene glycol 2000-10000, poloxamer 188, carnauba wax, hydrogenated castor oil, stearyl alcohol, cetyl alcohol and mixtures thereof. In order to achieve the desired release rates, other excipients such as lactose, microcrystalline cellulose, starch etc. can be added.

These pellets are then coated by retarding lacquers as described for the pellets consisting of inert starters with drug layers sprayed onto them.

Some excipients are suitable also to achieve extruded pellets with suitable extended release even without retarding lacquers. These are e.g. carnauba wax, hydrogenated castor oil and mixtures thereof for lipophilic pellets or carbopol, anionic carboxylic acrylic polymer e.g. partly methyl esterified methacrylic acid polymers. Suitable partly methyl esterified methacrylic acid polymers are sold under the names Eudragit L and Eudragit S, preferably used are Eudragit S100 and L100.

The extended release pellets can be of sizes between 0.2 and 3 mm in diameter, preferably between 0.5 to 1.5 mm, most preferred between 0.7 and 1.0 mm. According to the present invention the pellets are preferably filled in hard capsules. The extended release capsules can be of any size and shape and colour, e.g. for a 0.75 mg dose strengths preferably a size 3 capsule can be used. The capsule shell is usually made from hydroxypropyl methylcellulose (so-called HPMC or vegetable capsules) or gelatine. The capsules according to the present invention are usually filled with pellets, for example, more than 150 extended release pellets. Each pellet is built up of an inert (starter) core pellet, an active ingredient layer and an extended or slow release film coating. In one capsule, the amount of pramipexole or the pharmaceutically acceptable salt thereof contained in the pellets may preferably be sufficient to provide a daily dose administered at one time.

Alternatively the extended release pellets can be admixed with fillers and binders, such as microcrystalline cellulose, carrageenans, and alginates and disintegrants, such as sodium starch glycolate, sodium carboxymethylcellulose (croscarmellose), further excipients, like glidants and lubricants, and be compressed into tablets.

The present invention is further directed to the use of the extended release pellet formulation or capsule according to the present invention for preparing a medical composition in combination with another treatment for Parkinson's Disease and complications or disorders associated therewith.

The aforementioned pramipexole containing extended release formulations may be given in combination with a conventional treatment of Parkinson's Disease, the active ingredient of which is selected from at least one of:
anti-Parkinson's Disease anticholinergics,
COMT-inhibitor,
MAO-B-inhibitors,
Beta blockers,
DCC-inhibitors,
Acetylcholinersterase inhibitors.

Preferred are combinations of the aforementioned pramipexole tablets with at least one other active ingredient being selected from of:
L-DOPA,
L-DOPA in combination with entacapone, or tolcapone,
carbidopa
benserazid,
entacapone,
tolcapone,
amantadine,
selegiline,
rasagiline,
azilect,
deprenyl,
comtan,
propranolol,
safinamide,
donezepil.

Preferably the combination consists of at least two physically separated pharmaceutical formulations, one of which is the pramipexole comprising formulation and the second one of which is the formulations comprising the combination partner of the aforementioned group. It is not mandatory that the two combinations are taken at the same point in time or with the same frequency. The advantage of this free combination type is that the skilled person in the art, the physician, can adjust the dosages and frequencies of application of each combination partner separately and in accordance with the needs of the patients.

The dosage units (tablets, capsules etc.) of each component of the combination according to the present invention can be packaged in a container, accompanied by a package insert providing pertinent information such as, for example, dosage and administration information, contraindications, precautions, drug interactions and adverse reactions.

In an alternative embodiment the combinations can be offered in one package or are otherwise physically interconnected (kit or kit of parts) in order to facilitate the handling for the patient.

1. The use of an extended release tablet formulation comprising pramipexole or a pharmaceutically acceptable salt thereof in a matrix comprising at least one water swelling polymer other than pregelatinized starch in combination with a conventional treatment option of Parkinson's Disease for the manufacture of a medicament for the treatment of Parkinson's Disease, in particular advanced Parkinson's Disease.

2. The use of an extended release tablet formulation comprising pramipexole or a pharmaceutically acceptable salt thereof in a matrix comprising
(a) at least one water swelling polymer other than pregelatinized starch and optionally excipients, the resulting tablet providing a pH-independent in vitro release characteristic in the range from pH 1 to 7.5, or
(b) at least one water swelling anionic polymer and optionally excipients, the resulting tablet providing a pH-dependent release characteristic with a faster release characteristic in the range of pH<4.5, and a slower and further on pH-independent release characteristic in the range from pH 4.5 to 7.5,
in combination with a conventional treatment option of Parkinson's Disease for the manufacture of a medicament for the treatment of Parkinson's Disease.

3. The use according to claim 1 wherein the matrix of the pramipexole extended release tablet comprises at least one water swelling polymer other than pregelatinized starch, a water swelling anionic polymer and optionally excipients, the resulting tablet providing a pH-dependent release characteristic with a faster release characteristic in the range of pH<4.5, and a slower and further on pH-independent release characteristic in the range from pH 4.5 to 7.

4. The use of an extended release pellet comprising an active ingredient selected from pramipexole and the pharmaceutically acceptable salts thereof, and at least one release-modifying excipient in combination with a conventional treatment option of Parkinson's Disease for the manufacture of a medicament for the treatment of Parkinson's Disease, in particular advanced Parkinson's Disease.

5. The use of an extended release pellet comprising
an inert pellet core;
a first layer being an active ingredient layer comprising pramipexole or a pharmaceutically acceptable salt thereof and optionally one or more wet binders and further excipients; and
a second layer provided on the first layer, the second layer being an extended release coating comprising
(a) at least one water-insoluble polymer and optionally a pore former, the resulting pellet having a pH-independent in vitro release characteristic or
(b) a mixture of a pH-dependent enteric-coating polymer and a pH-independently water swelling polymer, the resulting pellet having a close to zero order in vitro release characteristic at acidic pH values up to pH 6.8, an accelerated release above pH 6.8 and a more accelerated release above pH 7.3
in combination with a conventional treatment option of Parkinson's Disease for the manufacture of a medicament for the treatment of Parkinson's Disease, in particular advanced Parkinson's Disease.

6. The use according to claim 1, wherein the conventional treatment option of Parkinson's Disease comprises a pharmaceutical composition containing at least one ingredient selected from the group of anticholinergics, COMT-inhibitors, MAO-B-inhibitors, beta blockers, DCC-inhibitors, acetylcholinesterase inhibitors each of which in a pharmacologically effective amount for to treat Parkinson's Disease.

7. The use according to claim 1, wherein the conventional treatment option of Parkinson's Disease comprises at least one ingredient selected from the group of carbidopa, benserazid, entacapone, tolcapone, amantadine, selegiline, rasagiline, azilect, deprenyl, comtan, propranolol, L-DOPA, L-DOPA in combination with entacapone or tolcapone, each of which in a pharmacologically effective amount for to treat Parkinson's Disease.

8. The use according to claim 1, wherein the conventional treatment option of Parkinson's Disease comprises at least one ingredient selected from the group of carbidopa, benserazid, entacapone, tolcapone, amantadine, selegiline, rasagiline, azilect, deprenyl, comtan, propranolol, L-DOPA in combination with entacapone or tolcapone, each of which in a pharmacologically effective amount for to treat Parkinson's Disease.

9. The use according to claim 1, wherein the conventional treatment option of Parkinson's Disease comprises at least one ingredient selected from the group of carbidopa, benserazid, entacapone, tolcapone, amantadine, selegiline, rasagiline, azilect, deprenyl, comtan, propranolol each of which in a pharmacologically effective amount for to treat Parkinson's Disease.

10. The use according to claim 1, wherein the conventional treatment option of Parkinson's Disease comprises at least one ingredient selected from the group of entacapone, tolcapone, amantadine, selegiline, rasagiline, azilect, deprenyl, comtan, propranolol each of which in a pharmacologically effective amount for to treat Parkinson's Disease.

11. The use according to claim 1, wherein the conventional treatment option of Parkinson's Disease comprises at least one ingredient selected from the group of safinamide, donezepil each of which in a pharmacologically effective amount for to treat Parkinson's Disease.

12. The use according to claim 1, characterised in that the pramipexole is comprised in a first pharmaceutical composition and the combination partner is comprised in a second pharmaceutical composition.

13. The use according to claim 12, characterised in that the two pharmaceutical compositions are packed in physically separated packages.

14. The use according to claim 12, characterised in that the two pharmaceutical compositions are packed in packages which are physically linked.
